(19) United States
(12) Patent Application Publication
(10) Pub. No.: US 2023/0263989 A1
(43) Pub. Date: Aug. 24, 2023

(54) Blood Pressure Controlling Device

(71) Applicants: Ali Rahmani, Fort Myers, FL (US); Patricia Rahmani, Fort Myers, FL (US)

(72) Inventors: Ali Rahmani, Fort Myers, FL (US); Patricia Rahmani, Fort Myers, FL (US)

(21) Appl. No.: 17/679,754

(22) Filed: Feb. 24, 2022

(51) Int. Cl. A61M 21/02

(52) U.S. Cl. CPC A61M 21/02; A61M 2210/0618

(57) ABSTRACT

A device to be worn in the nostrils of a user that induces unidirectional airflow through each nostril. This unidirectional airflow may be controlled by a pair of one-way check valves in the path of each nostril.

### CROSS-REFERENCES TO RELATED APPLICATIONS

Not Applicable.

### FIELD OF INVENTION

The present invention relates generally to an apparatus that may be used for managing blood pressure. More particularly, the present invention is related to device to aid in alternating nostril breathing techniques for blood pressure reduction.

### BACKGROUND OF THE INVENTION

Alternating nostril breathing practice during meditation has been demonstrated to be efficacious in health literature for reducing high blood pressure. Alternating nostril breathing refers to the practice of breathing in through one nostril and breathing out through the other. Traditionally, this is accomplished by closing one nostril with a finger on the in breath and switch to closing the other nostril with a finger on the out breath. While effective, this action can become tedious to do continuously and may interfere with achieving a properly meditative state.

There exists a need for an improved method of achieving alternative nostril breathing during meditation.

### SUMMARY OF THE INVENTION

This Summary is provided to introduce a selection of concepts in a simplified form that is further described below in the Detailed Description. This Summary is not intended to identify key features or essential features of the claimed subject matter, nor is it intended to be used as an aid in determining the scope of the claimed subject matter. The present invention is defined by the claims.

Embodiments of the present invention may comprise a device to be worn in the nostrils of a user that induces unidirectional airflow through each nostril. In some embodiments, this unidirectional airflow may be controlled by a pair of one-way check valves in the path of each nostril.

### BRIEF DESCRIPTION OF THE DRAWINGS

The present invention is described in detail below with reference to the attached drawing figures, wherein:

FIG. 1 provides an illustrative view of an exemplary embodiment in accordance with the present invention.

FIGS. 2A and 2B provide illustrative views of airflow through an exemplary embodiment in accordance with aspects of the present invention.

FIG. 3 provides an exploded view of an exemplary embodiment in accordance with aspects of the present invention.

FIG. 4 provides an exploded view of an exemplary embodiments in accordance with aspects of the present invention.

### DETAILED DESCRIPTION OF THE INVENTION

Some embodiments of the present invention are described with specificity herein to meet statutory requirements. However, the scope of the invention is not intended to be defined by the description itself. The claimed subject matter may be embodied as to include different features, elements, components, steps, or combinations of steps, similar to those described herein, and in combination with other existing or future technologies. Moreover, although the term “step” might be used to connote different elements of the methods employed, this term should not be interpreted as implying any particular order among or between various steps herein disclosed unless and except the order of individual steps is explicitly described or required.

Embodiments of the present invention may comprise a device to be worn in the nostrils of a user that induces unidirectional airflow through each nostril. In some embodiments, this unidirectional airflow may be controlled by a pair of one-way check valves in the path of each nostril.

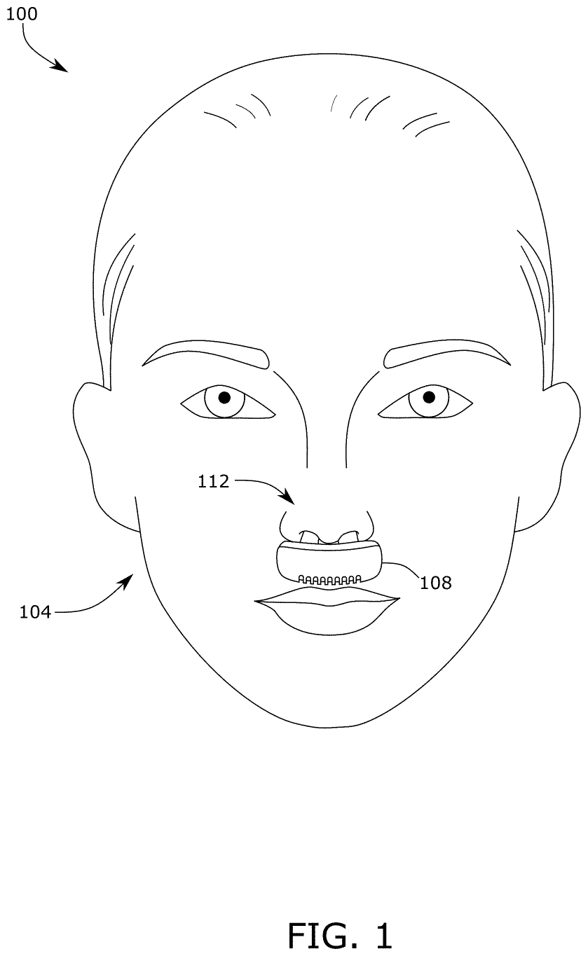

FIG. 1 provides an illustrative view 100 of an exemplary embodiment in accordance with the present invention. As depicted, user 104 wears device 108 in user's nose 112. Device 108 is configured such that it may remain suspended by user's nose 112 without requiring user 104 to hold device 108 in place. This may allow for greater comfort for user 104 and allow user 104 to achieve a more meditative state.

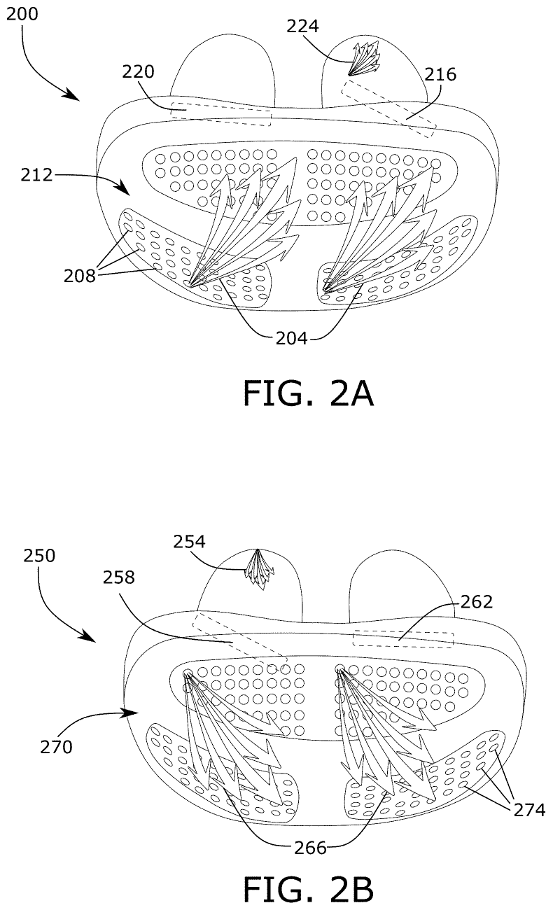

FIGS. 2A and 2B provide illustrative views 200 and 250 of airflow through an exemplary embodiment in accordance with aspects of the present invention. As depicted in FIG. 2A, on inhale, outside air 204 passes through vents 208 in device housing 212. The force of outside air 204 forces check valve 216 to open, while check valve 220 remains shut. This allows air 224 to selectively pass through device housing 212 into one nostril and not the other.

As depicted in FIG. 2B, on exhale, air 254 passes from a user's nostril (not shown). The force of air causes check valve 258 to open, while check valve 262 remains closed, resulting in air 254 selectively passing through one nostril and not the other. Air 266 then passes through device housing 270 via vents 274.

In some embodiments of the present invention, air filtration elements may be incorporated in the device housing to reduce particulate matter from passing between the outside world and the user's nostrils.

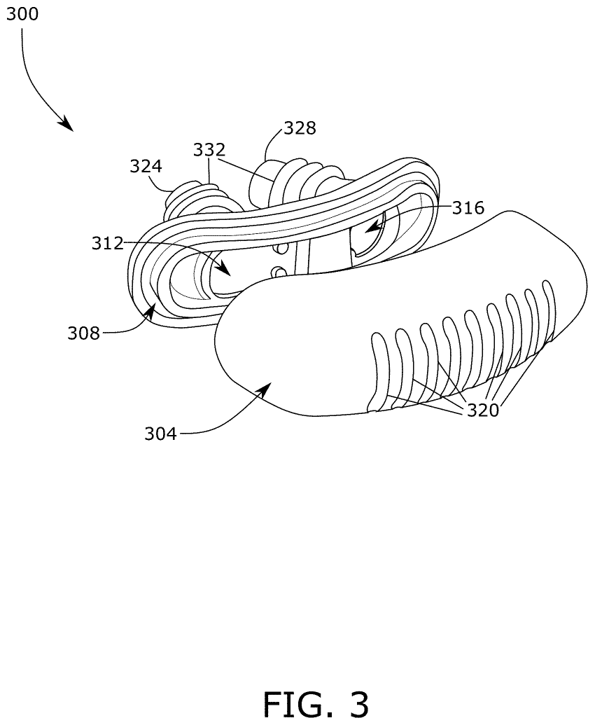

FIG. 3 provides an exploded view 300 of an exemplary embodiment in accordance with aspects of the present invention. As depicted, device housing 304 is shown removed from base 308 to expose check valves 312 and 316. Device housing 304 includes vents 320 to allow air to pass through. Base 308 include nasal inserts 324 and 328 that are configured to fit within a user's nostrils. Check valves 312 allows air to pass unidirectionally between nasal insert 324 and device housing 304 in a first direction, while check valves 316 allows air to pass unidirectionally between nasal insert 328 and housing 304 in the opposite direction. Nasal inserts 324 and 328 include a series of ribs 332 that are configured to snuggly fit within a user's nostrils (not shown) to improve control of air flow and ensure the device remains in place.

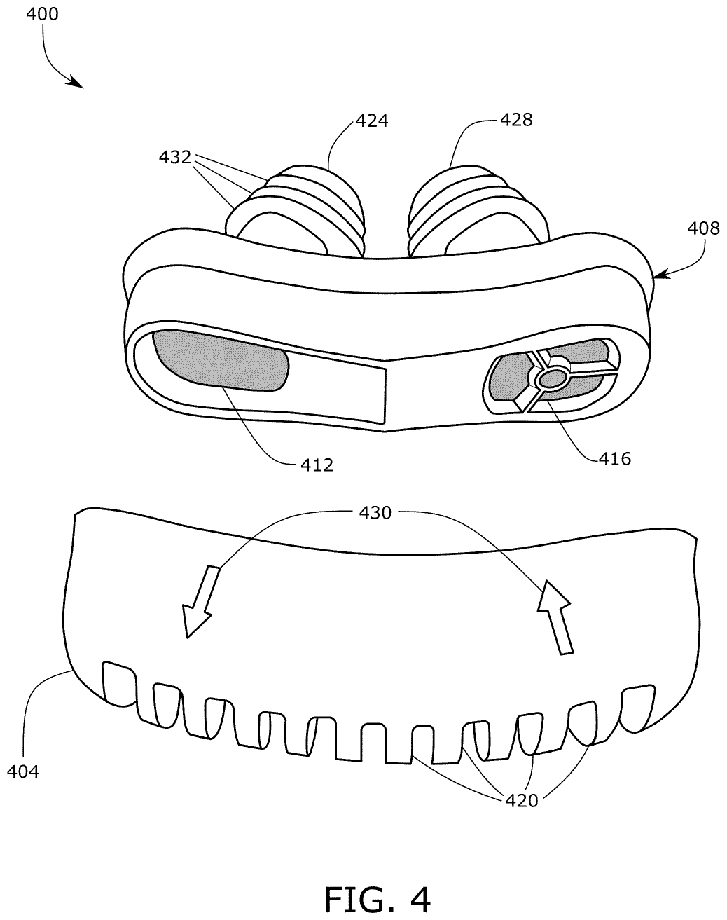

FIG. 4 provides an exploded view 400 of an exemplary embodiments in accordance with aspects of the present invention. As depicted, device housing 404 is shown removed from base 408 to expose check valves 412 and 416. Device housing 404 includes vents 420 to allow air to pass through. Base 408 include nasal inserts 424 and 428 that are configured to fit within a user's nostrils. Check valves 412 allows air to pass unidirectionally out from nasal insert 424 through device housing 404, while check valves 416 allows air to pass unidirectionally into nasal insert 428 from housing 404. Directional indicia 430 indicate the directions that each of the check valves 412 and 416 allow air to pass so that a user may properly orient the device when worn. Nasal inserts 424 and 428 include a series of ribs 432 that are configured to snuggly fit within a user's nostrils (not shown) to improve control of air flow and ensure the device remains in place.

Embodiments of the present invention have been described, as required by statute, to be illustrative, but should not be interpreted to be restrictive. One having skill in the art will recognize that many different arrangements of the various components depicted are possible without departing from the scope of the claims below, as well as arrangements including components not explicitly shown.

One having skill in the art will understand that certain combinations and/or sub-combinations of elements and features are of utility and may be employed without reference to other combinations and/or sub-combinations and are contemplated within the scope of the claims. Not all steps listed in the various figures need be carried out in the specific order described.

What is claimed is:

1. A blood pressure reducing devices comprising:
a device housing attached to a device base,
wherein the device housing includes one or more vents configured to allow air to pass through;
a first check valve and a second check valve attached to the device base and contained within the device housing;
a first nasal insert attached to the device base; and
a second nasal insert attached to the device base,
wherein the first and second nasal inserts are configured to fit within a user's nostrils,
wherein the first check valve allows air to pass unidirectionally between the first nasal insert and the device housing in a first direction, and
wherein the second check valve allows air to pass unidirectionally between the second nasal insert and the device housing in a second direction, the second direction being opposite to the first direction.

2. The blood pressure reducing devices of independent claim 1, wherein:
the first and the second nasal inserts include a series of ribs configured to snuggly fit within the user's nostrils to improve control of air flow and ensure the device remains in place.
